(19) United States
(12) Patent Application Publication
(10) Pub. No.: US 2020/0129778 A1
(43) Pub. Date: Apr. 30, 2020

(54) Micro-Solenoid Inductors With Magnetic Core for Neural Stimulation

(71) Applicant: Northeastern University, Boston, MA (US)

(72) Inventors: Nian Xiang Sun, Winchester, MA (US); Mohsen Zaeimbashi, Somerville, MA (US)

(21) Appl. No.: 16/664,557

(22) Filed: Oct. 25, 2019

Related U.S. Application Data
(60) Provisional application No. 62/750,341, filed on Oct. 25, 2018.

(51) Int. Cl. A61N 2/02; A61N 2/00

(52) U.S. Cl. CPC A61N 2/02; A61N 2/006

(57) ABSTRACT

A method of stimulating a neural cell may comprise disposing a neural stimulation probe substantially adjacent to the neural cell, and applying a signal to the neural stimulation probe. The neural stimulation probe may comprise a micro-coil, an input lead, an output lead, and a magnetic core. The micro-coil may comprise N of windings having a first end and a second end, with the input lead electrically coupled to the first end and the output lead coupled to the second end. The micro-coil may have a width that is less than or equal to 40 μm, a thickness of less than or equal to 20 μm, and a length of less than or equal to 80 μm. The magnetic core may be disposed such that the N windings are wrapped about the magnetic core. The micro-coil may be configured and arranged to generate a E-field gradient in the direction of the neural cell.

### RELATED APPLICATIONS

This application claims the benefit of U.S. Provisional Application No. 62/750,341, filed on Oct. 25, 2018. The entire teachings of the above application are incorporated herein by reference.

### GOVERNMENT SUPPORT

This invention was made with government support under Grant No. ECCS1533484 from the National Science Foundation. The government has certain rights in the invention.

### BACKGROUND

Brain neural stimulation may effectively treat a wide range of neurological disorders such as, for example, Parkinson disease, major depression, and stroke. Micro-electrode based devices are widely used for brain neural modulation and have been demonstrated to be effective and efficient for this purpose. Electrode-based devices, however, may exhibit certain inherent problems and limitations. For example, micro-electrodes may not be reliable in terms of providing a consistent response over time. This lack of reliability may be due to the direct contact of electrodes with extracellular membranes, which results in reduction and oxidation at the electrode-tissue interface, thereby increasing the electrode impedance and changing the associated stimulation threshold. In addition, the direct charge exchange between electrode and tissue may damage the tissue cells due to an imperfect contact.

### SUMMARY

Magnetic stimulation using micro-solenoid inductors (also referred to herein as micro-coils) has been demonstrated as an effective method of brain neural modulation. Micro-coils may be superior to micro-electrodes for such biological applications in terms of long-term functionality, because, unlike electrode-based devices, micro-coils use magnetic fields for neural stimulation, and so do not require direct contact with brain tissues. The micro-coil may be entirely encapsulated and hermetically sealed by a bio-compatible material, thereby isolating the micro-coils from the relevant biological environment. The efficiency and stimulation capability of such magnetic coils therefore exhibit little or no degradation over time. Further, magnetic fields exhibit high permeability with respect to biological tissues, as compared to electric fields and acoustic energy.

The described embodiments of a micro-coil may be used to apply a small and precise magnetic field to a specific location to, for example, affect neural stimulation. A magnetic core associated with the micro-coil may be used to facilitate such a precisely directional magnetic field. The magnetic core may be magnetized to act as a permanent magnet, so that the resulting magnetic field comprises not only a field component stemming from an electrical current passing through the micro-coil, but also a field component stemming from the magnetized magnetic core.

In one aspect, an embodiment of the invention may be a neural stimulation probe, comprising a micro-coil, an input lead, an output lead, and a magnetic core. The neural stimulation probe may comprise N windings having a first end and a second end. The micro-coil may have a width that is less than or equal to 40 μm and a length of less than or equal to 80 μm. The input lead may be electrically coupled to the first end of the micro-coil, and the output lead may be electrically coupled to the second end of the micro-coil. The magnetic core may be disposed such that the N windings are wrapped about the magnetic core.

The micro-coil may have a circular cross-section with a diameter that is less than or equal to 40 μm. The micro-coil may have a rectangular cross-section with a width that is less than or equal to 40 μm and a thickness that is less than or equal to 20 μm.

The neural stimulation probe may further comprise a bio-compatible material disposed about the micro-coil, the magnetic core, the first lead, and the second lead, such that the micro-coil, the magnetic core, the first lead, and the second lead are hermetically sealed within the bio-compatible material. The micro-coil may be configured to generate a first E-field oriented in a first direction, and a second E-field and a third E-field oriented in directions orthogonal to the first direction, and wherein the first E-field is substantially larger than the second and third E-fields. The micro-coil may be configured to generate a first E-field gradient in a first direction, and a second E-field gradient and a third E-field gradient both oriented in directions orthogonal to the first direction. The first E-field gradient may be substantially larger than the orthogonal E-field gradients.

The micro-coil, the magnetic core, the first lead, and the second lead may be fabricated on a silicon shank. A bio-compatible material may be disposed about the micro-coil, the magnetic core, the first lead, the second lead, and the shank, such that the micro-coil, the magnetic core, the first lead, the second lead, and the shank are hermetically sealed within the bio-compatible material. The number of windings, N, may be substantially equal to six.

The magnetic core may comprise a material having both a substantial relative permeability and a substantial magnetization factor. The substantial relative permeability may be at least 800. The magnetic core may comprise either FeGaB or NiFe.

In another aspect, an embodiment of the invention may be a method of stimulating a neural cell, comprising disposing a neural stimulation probe substantially adjacent to the neural cell and applying a signal to the neural stimulation probe. The neural stimulation probe may comprise a micro-coil that has N windings. The N windings may have a first end and a second end. The micro-coil may have a width that is less than or equal to 40 μm, a thickness of less than or equal to 20 μm, and a length of less than or equal to 80 μm. The neural stimulation probe may further comprise an input lead electrically coupled to the first end of the micro-coil, an output lead electrically coupled to the second end of the micro-coil, and a magnetic core disposed such that the N windings are wrapped about the magnetic core. Applying a signal to the neural stimulation probe may further comprise applying a signal to the microcoil through the input lead and the output lead.

The method may further comprise hermetically sealing the micro-coil, the magnetic core, the first lead, and the second lead by disposing a bio-compatible material about the micro-coil, the magnetic core, the first lead, and the second lead.

The method may further comprise generating, by the micro-coil, a first E-field oriented in a first direction, and a second E-field and a third E-field oriented in directions orthogonal to the first direction, and wherein the first E-field is substantially larger than the orthogonal E-fields. The method may further comprise generating, by the micro-coil, a first E-field gradient oriented in a first direction, and a second E-field gradient and a third E-field both oriented in directions orthogonal to the first direction, and wherein the first E-field is substantially larger than the orthogonal E-fields.

The method may further comprise arranging the neural stimulation probe so that the neural cell is in the first direction with respect to the micro-coil. The method may further comprise fabricating the micro-coil, the magnetic core, the first lead, and the second lead on a silicon shank. The method may further comprise hermetically sealing the micro-coil, the magnetic core, the first lead, the second lead, and the silicon shank by disposing a bio-compatible material about the micro-coil, the magnetic core, the first lead, the second lead, and the silicon shank.

Applying a signal to the micro-coil may further comprise applying an alternating current signal. The alternating current signal may comprise a half-cycle alternating current at about 100 mA and at about 13 MHz. The method may further comprise configuring the micro-coil as a solenoid coil.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A and 1B show an example embodiment of a micro-coil according to the invention.

FIGS. 2A and 2B show another example embodiment of a micro-coil according to the invention.

FIGS. 3A and 3B show another example embodiment of a micro-coil according to the invention.

FIGS. 4A-4D, 5A-5E, and 6 are plots of simulation results associated with the micro-coil configurations depicted in FIGS. 1A, 1B, 2A, 2B, 3A, and 3B.

### DETAILED DESCRIPTION

A description of example embodiments follows.

The teachings of all patents, published applications and references cited herein are incorporated by reference in their entirety.

The embodiments described herein include several examples of a magnetic neural stimulation probe according to the invention. Each magnetic neural stimulation probe comprises a micro-coil, the size of which may affect its performance. As the micro-coil is made smaller, its achievable spatial resolution (with respect to the neural stimulation process) increases. An embodiment of the micro-coil may be fabricated on a silicon shank or probe, and be inserted into a certain part of the brain for neural stimulation and investigation. A smaller micro-coil, and thus a smaller probe, will cause less damage to the brain tissue.

A micro-coil according to the described embodiments may comprise one or more of several different structures and geometries, such as, for example solenoid, spiral, and toroidal, among other such configurations. Parameters that relate to the inductance of a coil may include, for example (i) number of turns, (ii) material of the coil's core, (iii) cross-sectional area of the coil, and (iv) the end-to-end length of the coil. Of these parameters, the number of turns and the cross-sectional area may have hard limits to maintain a physically small micro-coil. Enhanced micro-coil inductance and performance may be attained by, for example, using a high-permeability magnetic material for the coil's core rather than an air core.

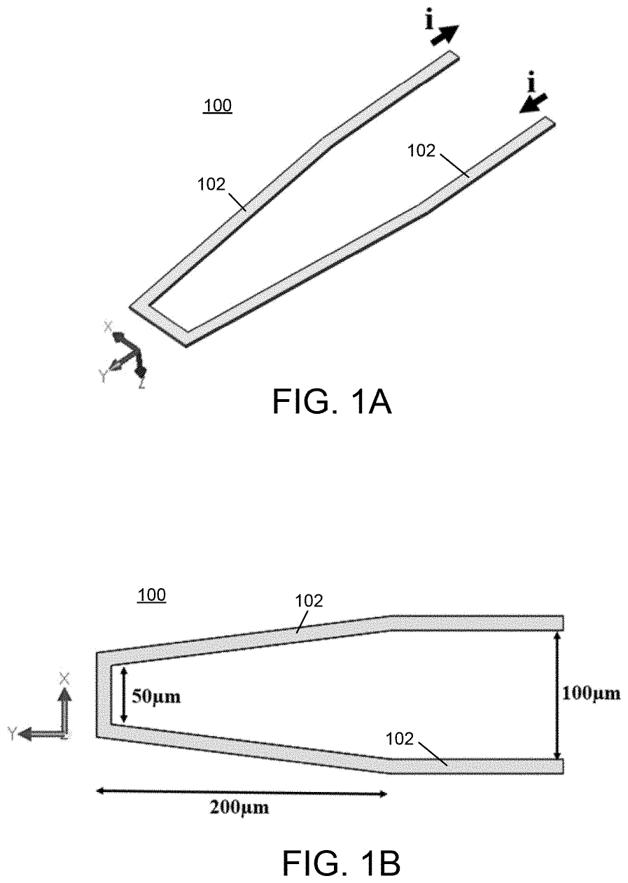

FIGS. 1A and 1B show an example embodiment of a micro-coil according to the invention, referred to herein as a flat coil 100, which is a structure comprising a single wire 102 that may be micro-fabricated on a silicon shank as an injectable probe. The geometry and dimensions of this flat coil are shown in FIGS. 1A and 1B. The specific dimensions shown are for example only, and are not intended to be limiting. For example, although the width of the tip of the flat coil 100 is shown in FIG. 1B as 50 nm, the tip width may be in the range of 10 nm to 100 μm. The distance between the input wires of the flat coil is shown as 100 μm, but that distance may be in the range of 100 nm to 500 μm. The length of the flat coil (from tip to input) is shown as 200 μm, but may be a length in the range of 50 μm to 1 mm.

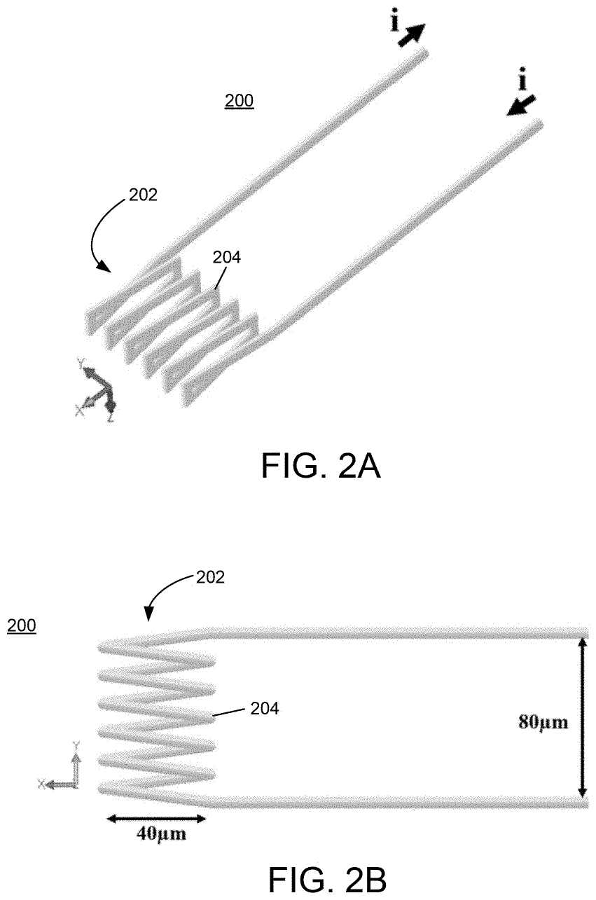

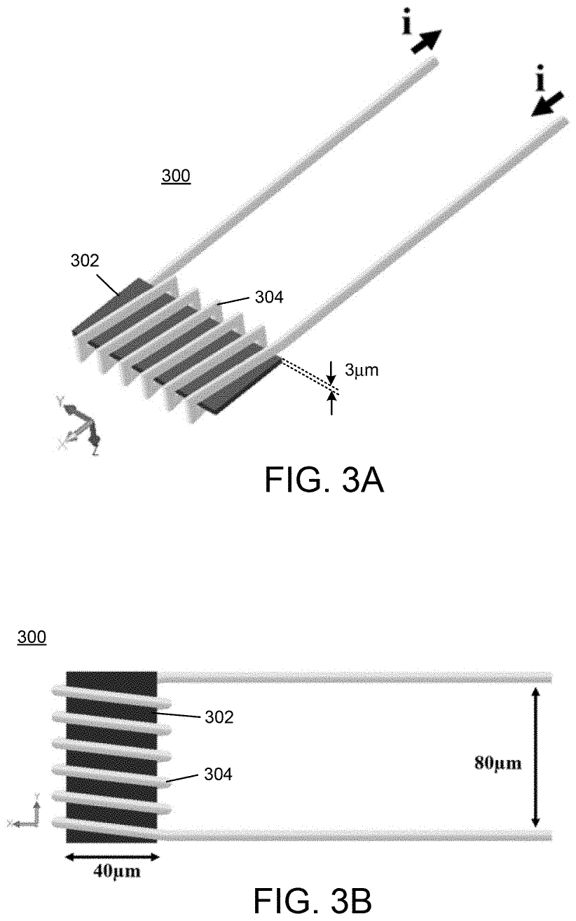

FIGS. 2A, 2B, 3A, and 3B show two other example embodiments of a micro-coil according to the invention, referred to herein as a solenoid inductor or solenoid coil. The example micro-coil 200 of FIGS. 2A and 2B is shown with an air core 202 and wire windings 204 wrapped about the air core 202.

The example micro-coil 300 of FIGS. 3A and 3B is shown with a FeGaB core 302 and a number N of wire windings 304 wrapped about the FeGaB core. In the example embodiment, FeGaB is a composite magnetic material with a high relative permeability μᵣ, although other materials with a high relative permeability, such as NiFe (also referred to as Permalloy), may alternatively be used for the core. Representative values of relative permittivity, relative permeability, and electrical conductivity of FeGaB are 1.0, 990, and 1.2E6 S/m, respectively, although the relative permeability of FeGaB may be different depending on the deposition process, and could be as high as 1300. These representative values are examples provided for descriptive purposes only, and are not intended to be limiting. As is known in the art, the parameters associated with the FeGaB material may be different from those set forth above, depending on the specific fabrication and deposition processes involved.

The example micro-coil 300 shown in FIGS. 3A and 3B has a coil structure with a rectangular cross-section, with a length of 80 μm, a width of 40 μm, and a thickness of 20 μm. It should be understood, however, that other embodiments may have a coil structure with a circular or other shape cross-section. An example micro-coil with a coil structure having a circular cross-section may have a length of 80 μm and an outer coil diameter of 40 μm (the outer coil diameter corresponding to the width of the rectangular cross-section coil structure). The specific dimensions shown are for example only, and are not intended to be limiting. For example, the length is shown as 80 μm, but may be a length in the range of 50 nm to 500 μm. The width of the input of the flat coil is shown as 100 μm, but may be a width in the range of 100 nm to 500 μm. The length of the flat coil (from tip to input) is shown as 200 μm, but may be a length in the range of 50 μm to 1 mm.

In this example embodiment, the number of turns for each solenoid coil 200, 300, is 6. The thickness of the FeGaB core 302 in FIGS. 3A and 3B is 3 μm, although this value is presented as an example only and is not intended to be limiting. The thickness may be within a range of 10 nm to 10 μm, although that range is not intended to be limiting. The wire winding material in all three coils 100, 200, and 300 is copper, although other electrically conductive materials may alternatively be used to implement the windings.

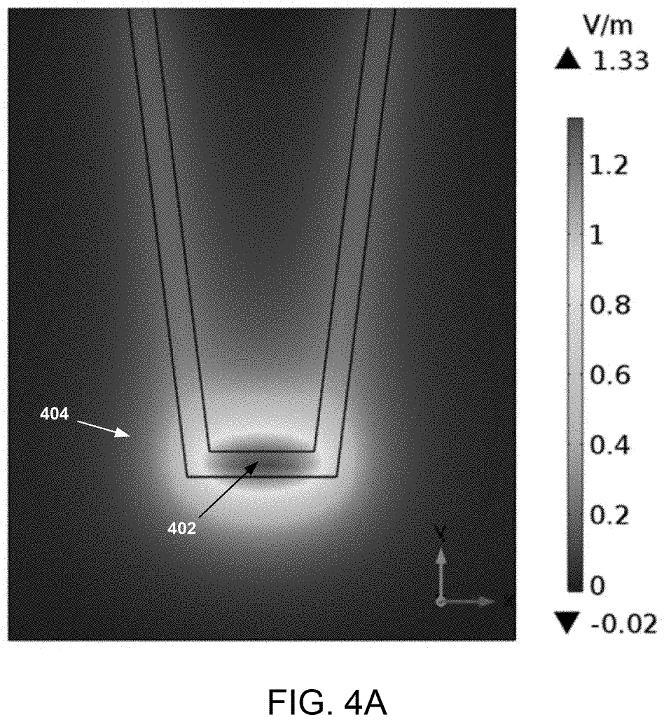

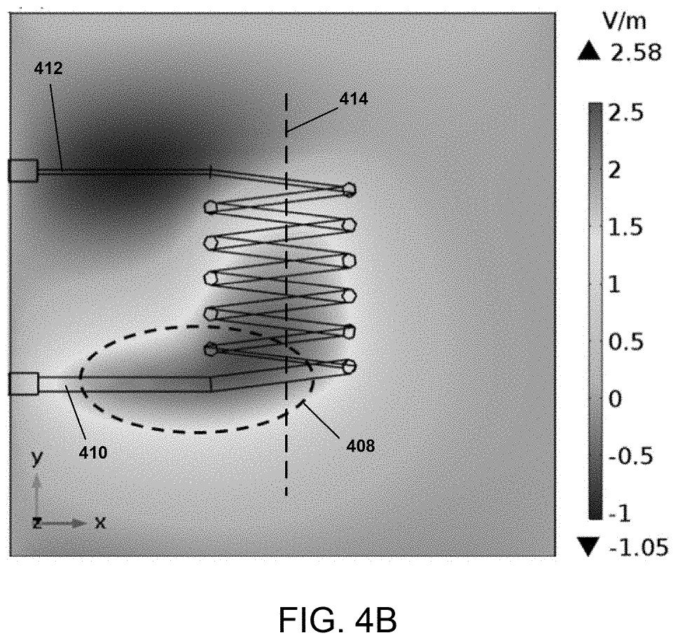

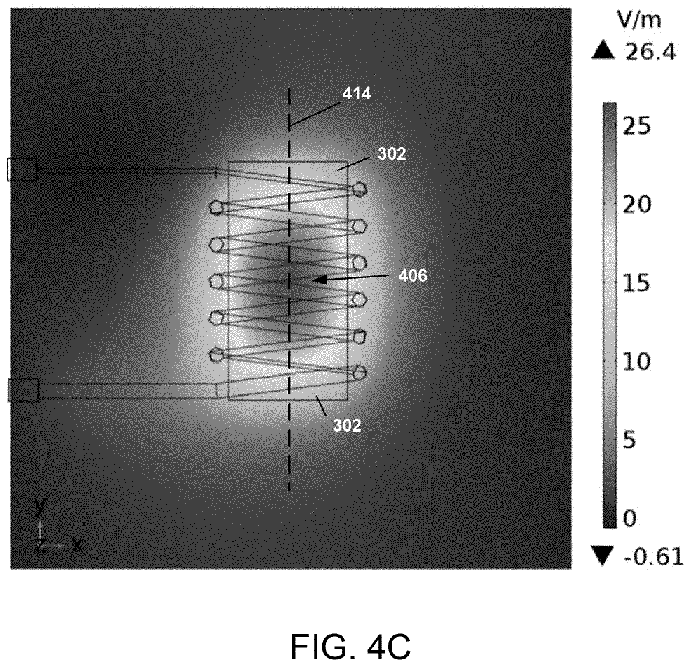

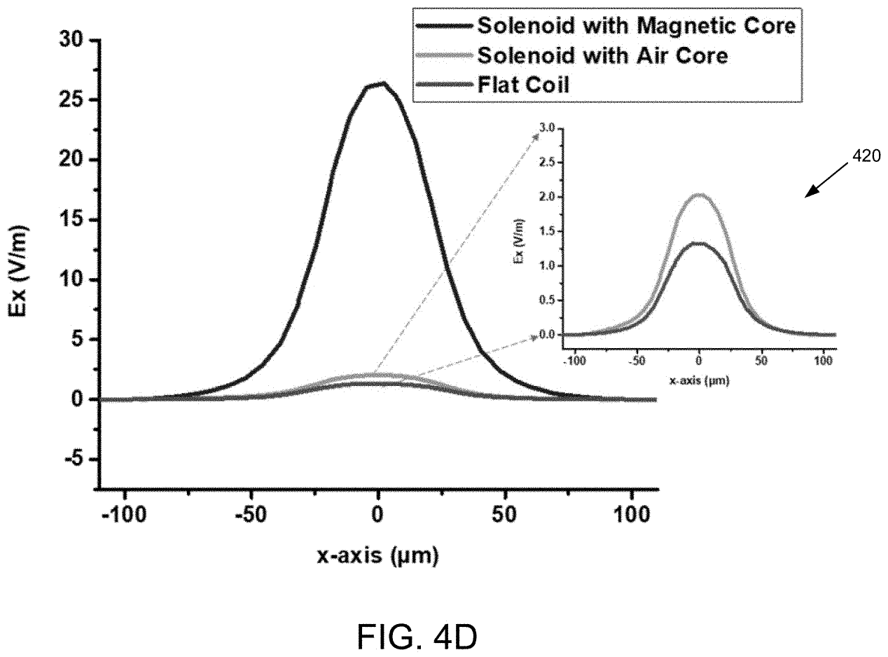

FIGS. 4A-4D, 5A-5E, and 6 are plots of simulation results associated with the micro-coil configurations depicted in FIGS. 1A, 1B, 2A, 2B, 3A, and 3B. The simulation applies a half-cycle alternating current at 100 ma and 13 MHz, although these values were selected for descriptive purposes and are not intended to be limiting. The alternating current may be half cycle or full cycle sinusoidal, or other cyclic waveforms. The alternating current may be within the range of 500 μa to 500 ma, and may have a cyclic frequency within the range of 1 Mhz to 100 MHz, although those ranges are not intended to be limiting.

While the strength of electric and magnetic fields (E and B) generated by a micro-coil are typically been evaluated when investigating the neural stimulation effect of the micro-coil, recent studies suggest that a more important parameter for magnetic stimulation may be the strength of electric E-field gradient (dEₓ/dx, dE_{y}/dy, dE_{z}/dz), i.e., the rate of change of an electric field component in respect to the corresponding axis.

To produce directionally selective neural stimulation with high resolution, embodiments of a micro-coil may be configured to generate a very high E-field gradient in a first direction, and very low gradient in the two orthogonal directions (with respect to the first direction). Such a micro-coil may be placed and oriented with respect to a target neural cell so that the target neural cell is selectively stimulated without disturbing other neural cells near the target neural cell. Specific techniques for configuring a micro-coil to generate a high E-field gradient in a first direction, and very low gradient in the two orthogonal directions, are known in the art and are beyond the scope of the present description.

The embodiments described herein focus on the results of an electric field and electric field gradient oriented in the x-direction, which is the direction of the field maximum. It should be understood, however, that the choice of the x-direction is for descriptive purposes only, and is not meant to be limiting. FIGS. 4A-4C show the Eₓ field component for three different micro-coils. FIG. 4A shows the Eₓ field component distribution corresponding to the micro-coil depicted in FIGS. 1A and 1B. The geometry of the coil is depicted by solid black lines. FIG. 4B shows the Eₓ field component distribution corresponding to the micro-coil depicted in FIGS. 2A and 2B (air core). FIG. 4C shows the Eₓ field component corresponding to the micro-coil depicted in FIGS. 3A and 3B (high relative permeability magnetic core). In all FIGS. 4A, 4B and 4C, the Eₓ distribution is shown in the plane defined by z=10 μm.

For the flat-coil configuration, the Eₓ field component is maximized on the central portion 402 of the tip 404 of, as shown in FIG. 4A.

For the solenoid coil configuration with a magnetic core, the Eₓ field component is maximum in the middle 406 of the coil, as shown in FIG. 4C.

For the solenoid coil configuration with an air core, the maximum Eₓ field component exists on the area 408 near the input wire 410, as shown in FIG. 4B. The input wire 410 of micro-coil is close to the z=10 μm plane, and the area 408 represents the electric field induced by magnetic flux around the input wire 410. Magnetic flux on this plane is along y-axis and therefore the induced electric field is maximized along x-axis. Because FIG. 4C depicts field distribution in the plane defined by z=10 μm, a substantial field is shown about the input wire 410. If the figure depicted field distribution at the z=−10 μm plane, the maximum Eₓ component would be shown above the output wire 412 rather than the input wire 410, because the output wire 412 is close to the z=−10 μm plane. The induced Eₓ field component is not as strong in the winding region of the micro-coil, because the magnetic flux is not directed along the y-axis due to angle of the winding wires.

FIG. 4D shows the Eₓ components for different coils along x-axis. For the flat coil, the center of the axis or x=0 is the middle 402 of the tip area 404 in FIG. 4A. For the solenoid coil, x=0 is the middle 414 of the coil as shown in FIGS. 4B and 4C. The inset 420 shows the expanded view of the flat-coil configuration and the air core solenoid coil configuration of the micro-coil. As shown, the Eₓ field component is more than 15 times larger in the high μᵣ magnetic core solenoid coil configuration, relative to either the flat-coil configuration or the air core solenoid coil configuration.

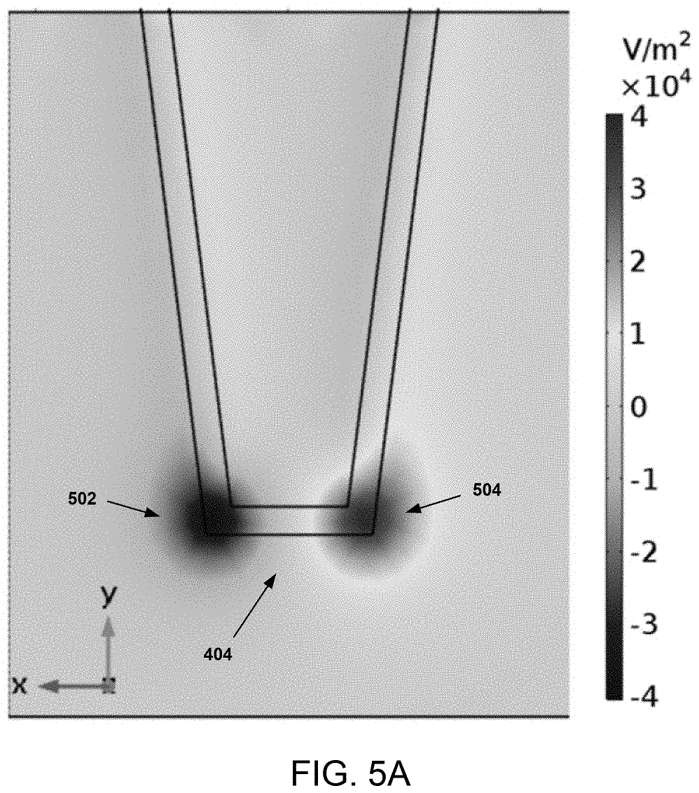

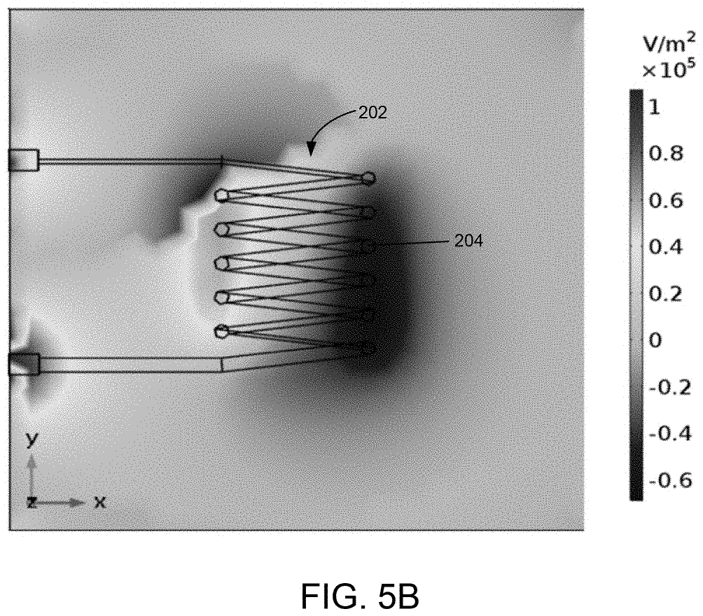

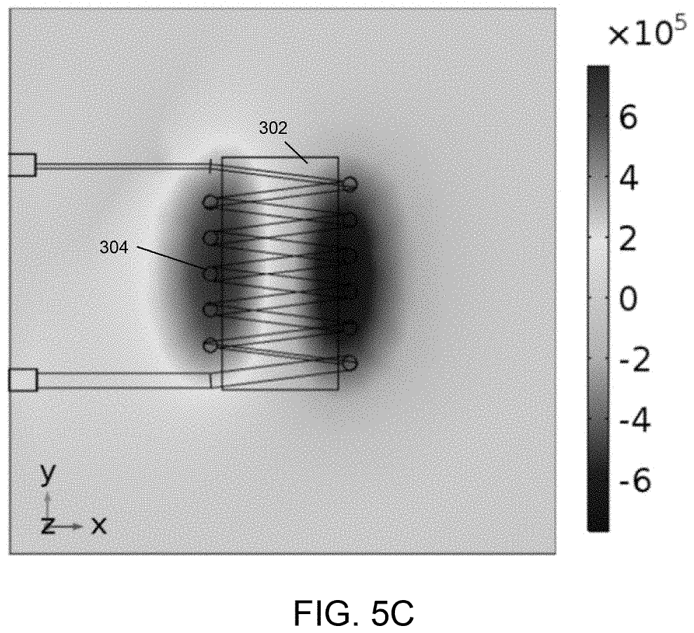

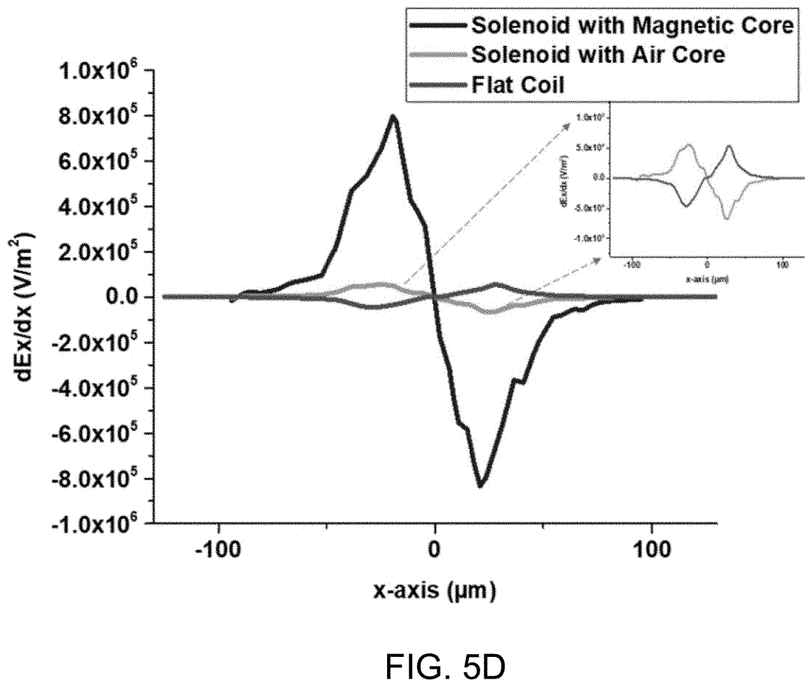

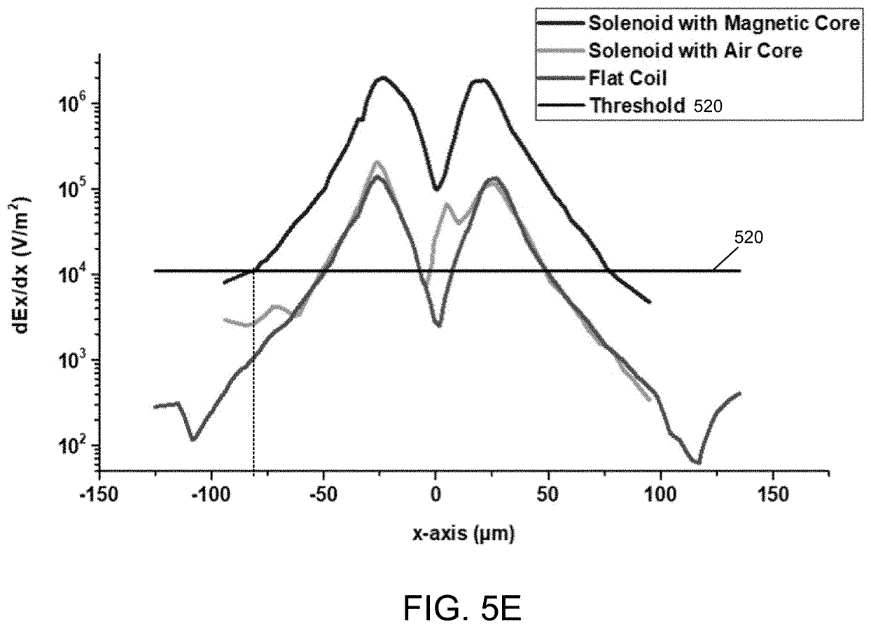

FIGS. 5A through 5E show the electric field gradient (dEₓ/dx) results, at the z=10 μm plane, for the flat-coil configuration, the high μᵣ core solenoid coil configuration, and the air core solenoid coil configuration.

FIG. 5A shows the dEₓ/dx for flat coil in which there are two maxima, one occurring on each of the corners 502, 504 of the flat coil tip 404. As can be seen in FIG. 5A, the rate of change of electric field at those two points is rapid.

FIGS. 5B and 5C show the dEₓ/dx in the air core solenoid coil and the high μᵣ magnetic core solenoid coil, respectively. Comparing FIGS. 5A through 5C, to FIGS. 4A through 4C, shows that the electric field gradient (dEₓ/dx) is zero in the area with constant Eₓ, and it is maximum in the area in which Eₓ is changing its value from maximum to minimum.

FIG. 5D shows the gradient dEₓ/dx along x-axis at the z=10 μm plane. As described with respect to FIG. 4D, x=0 occurs at the middle 402 of the tip 404 in the flat-coil configuration, and the middle 414 of the coil in the solenoid configuration.

FIG. 5E shows the gradient dEₓ/dx at the z=2 μm plane, on a logarithmic axis for a better visualization of the gradient, along with a threshold value 520 required for neural cell stimulation. As FIG. 5E shows, the field gradient is above the threshold value for all three coils, although the dEₓ/dx gradient value is at least an order of magnitude higher in the solenoid coil with high magnetic core than in the two other coils, and remains above the threshold up to about 80 μm away from the neural cell.

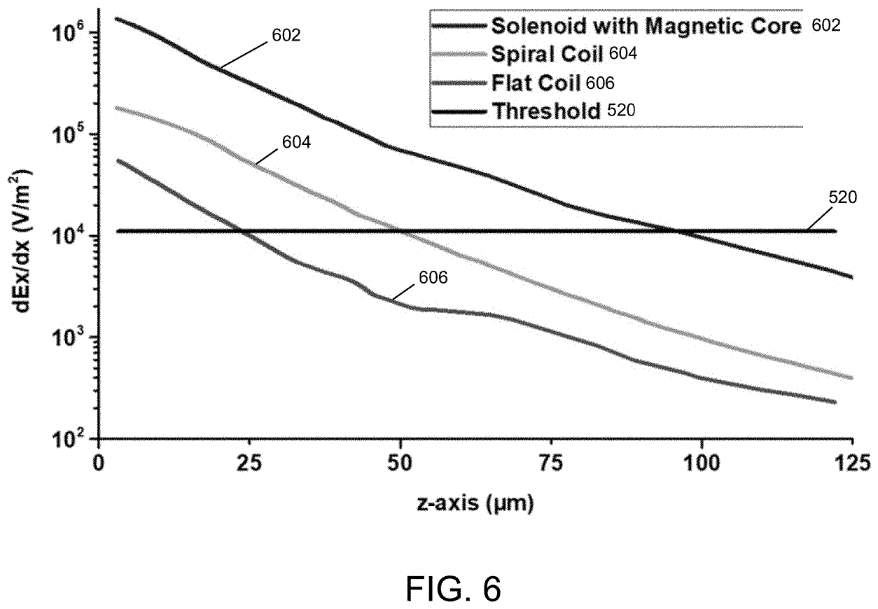

FIG. 6 shows the gradient dEₓ/dx along z-axis, which provides a relationship between distance from the coil and the threshold value 520. FIG. 6 thus conveys how far from the coil that the gradient dEₓ/dx remains above the threshold value 520. In other words, FIG. 6 shows how far from the neural cell the micro-coil can be located, while still affecting an efficient stimulation of the neural cell. As shown, the solenoid coils with a high μᵣ magnetic core (FeGaB in this example) is about four times more efficient than the flat coil, and two times more efficient than the solenoid with the air-core.

As demonstrated above, using a magnetic core with a high relative permeability can substantially improve the coil's performance by generating a higher magnetic and induced electric field, and therefore a higher electric field gradient. Using such a core material to amplify the fields may reduce power consumption and improve the micro-coil size factor.

Fields are amplified through the use of a magnetic core because the core material has a high Magnetization (M) factor. Magnetization, which is given by

$$M = \frac{{m}_{net}}{v} ,$$

is the net magnetic moment per unit volume in a bulk material. For the air core solenoid coil, the generated magnetic flux inside the coil is B₀∝μ₀nI=μ₀H, in which n is the number of turns and I is applied current. If the solenoid coil has a magnetic core, however, the generated magnetic flux in the interior of the coil is B=B₀+Bₘ, where Bₘ=μ₀M, which is the field component stemming from Magnetization of the core material (i.e., the core material acting as a permanent magnet). Therefore, total magnetic flux may be given by:

B=μ₀(H+M), (7)

where H is the field generated by the current flowing into the solenoid, and M is the intrinsic field inside the material. Magnetization may also be expressed by M=_{χ}H, where χ is referred to as magnetic susceptibility. Magnetic susceptibility describes how a material responds to an external field and how easy the material could be magnetized. Therefore, the final equation for generated magnetic flux could be written as follows:

B=μ₀(I+_{χ})H=μ₀μₓH (7)

The parameter μᵣ is called the relative permeability of the material and may be expressed as μᵣ=I+_{χ}. The magnetic flux inside a magnetic core solenoid coil may be amplified by a factor Using a high permeability material like Permalloy (NiFe) as the core material, the flux density of the interior region of the micro-coil may be enhanced by a factor of several thousand.

While example embodiments have been particularly shown and described, it will be understood by those skilled in the art that various changes in form and details may be made therein without departing from the scope of the embodiments encompassed by the appended claims.

What is claimed is:

1. A neural stimulation probe, comprising:
a micro-coil comprising N windings, the N windings having a first end and a second end, the micro-coil having a width that is less than or equal to 40 μm and a length of less than or equal to 80 μm;
an input lead electrically coupled to the first end of the micro-coil;
an output lead electrically coupled to the second end of the micro-coil; and
a magnetic core disposed such that the N windings are wrapped about the magnetic core.

2. The neural stimulation probe of claim 1, wherein the micro-coil has a circular cross-section with a diameter that is less than or equal to 40 μm.

3. The neural stimulation probe of claim 1, wherein the micro-coil has a rectangular cross-section with a width that is less than or equal to 40 μm and a thickness that is less than or equal to 20 μm.

4. The neural stimulation probe of claim 1, further comprising a bio-compatible material disposed about the micro-coil, the magnetic core, the first lead, and the second lead, such that the micro-coil, the magnetic core, the first lead, and the second lead are hermetically sealed within the bio-compatible material.

5. The neural stimulation probe of claim 1, wherein the micro-coil is configured to generate a first E-field oriented in a first direction, and a second E-field and a third E-field oriented in directions orthogonal to the first direction, and wherein the first E-field is substantially larger than the second and third E-fields.

6. The neural stimulation probe of claim 1, wherein the micro-coil is configured to generate a first E-field gradient in a first direction, and a second E-field gradient and a third E-field gradient both oriented in directions orthogonal to the first direction, and wherein the first E-field gradient is substantially larger than the orthogonal E-field gradients.

7. The neural stimulation probe of claim 1, wherein the micro-coil, the magnetic core, the first lead, and the second lead are fabricated on a silicon shank.

8. The neural stimulation probe of claim 7, wherein a bio-compatible material is disposed about the micro-coil, the magnetic core, the first lead, the second lead, and the shank, such that the micro-coil, the magnetic core, the first lead, the second lead, and the shank are hermetically sealed within the bio-compatible material.

9. The neural stimulation probe of claim 1, wherein N is substantially equal to six.

10. The neural stimulation probe of claim 1, wherein the magnetic core comprises a material having both a substantial relative permeability and a substantial magnetization factor.

11. The neural stimulation probe of claim 10, where the substantial relative permeability is at least 800.

12. The neural stimulation probe of claim 1, wherein the magnetic core comprises either FeGaB or NiFe.

13. A method of stimulating a neural cell, comprising:
disposing a neural stimulation probe substantially adjacent to the neural cell, the neural stimulation probe comprising:
a micro-coil comprising N windings, the N windings having a first end and a second end, the micro-coil having a width that is less than or equal to 40 μm, a thickness of less than or equal to 20 μm, and a length of less than or equal to 80 μm;
an input lead electrically coupled to the first end of the micro-coil;
an output lead electrically coupled to the second end of the micro-coil; and
a magnetic core disposed such that the N windings are wrapped about the magnetic core;
applying a signal to the microcoil through the input lead and the output lead.

14. The method of claim 13, further comprising hermetically sealing the micro-coil, the magnetic core, the first lead, and the second lead by disposing a bio-compatible material about the micro-coil, the magnetic core, the first lead, and the second lead.

15. The method of claim 13, further comprising generating, by the micro-coil, a first E-field oriented in a first direction, and a second E-field and a third E-field oriented in directions orthogonal to the first direction, and wherein the first E-field is substantially larger than the orthogonal E-fields.

16. The method of claim 13, further comprising generating, by the micro-coil, a first E-field gradient oriented in a first direction, and a second E-field gradient and a third E-field both oriented in directions orthogonal to the first direction, and wherein the first E-field is substantially larger than the orthogonal E-fields.

17. The method of claim 14, further comprising arranging the neural stimulation probe so that the neural cell is in the first direction with respect to the micro-coil.

18. The method of claim 13, further comprising fabricating the micro-coil, the magnetic core, the first lead, and the second lead on a silicon shank.

19. The method of claim 13, further comprising hermetically sealing the micro-coil, the magnetic core, the first lead, the second lead, and the silicon shank by disposing a bio-compatible material about the micro-coil, the magnetic core, the first lead, the second lead, and the silicon shank.

20. The method of claim 13, wherein applying a signal to the micro-coil further comprises applying an alternating current signal.

21. The method of claim 20, wherein the alternating current signal comprises a half-cycle alternating current at about 100 mA and at about 13 MHz.

22. The method of claim 13, further comprising configuring the micro-coil as a solenoid coil.
